(19) United States
(12) Patent Application Publication
(10) Pub. No.: US 2008/0182877 A1
(43) Pub. Date: Jul. 31, 2008

(54) Rimonabant forms and methods of making the same

(76) Inventor: Raymond J.H. Westheim, Oss (NL)

Correspondence Address: SYNTHON IP INC, 7130 HERITAGE VILLAGE PLAZA, STE 202, GAINESVILLE, VA, 20155, US

(21) Appl. No.: 12/004,738

(22) Filed: Dec. 21, 2007

Related U.S. Application Data
(60) Provisional application No. 60/883,697, filed on Jan. 5, 2007.

(51) Int. Cl. C07D 401/02; A61K 31/454

(52) U.S. Cl. 514/326; 546/211

(57) ABSTRACT

Various solid state forms of rimonabant have been found, including a particulate amorphous form as well as several hydrates and solvates.

This application claims the benefit of priority under 35 U.S.C. § 119(e) from U.S. Provisional Application Ser. No. 60/883,697, filed Jan. 5, 2007, the entire contents of which are incorporated herein by reference.

### BACKGROUND OF THE INVENTION

The present invention relates to rimonabant forms, methods of making the same, and pharmaceutical compositions containing the same.

Rimonabant of formula (1)

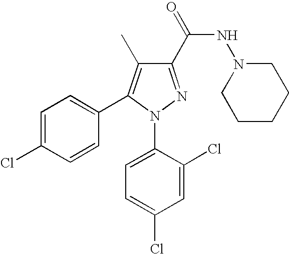

is disclosed in U.S. Pat. No. 5,624,941 (and corresponding EP 0,656,354) and is a pharmaceutically active compound that acts as a selective antagonist of cannabinoid type 1 (CB 1) receptor. It has been approved for use in a pharmaceutical product in Europe for the treatment of obesity. In the pharmaceutical product, rimonabant is present as a free base. The pharmaceutical product including the free base rimonabant is an immediate release film coated tablet having a 20 mg strength.

WO 03/040105 A1 (corresponding to US 2005/0043356 A1 and EP 1,446,384) discloses the existence of two polymorphic forms of crystalline rimonabant base, designated as Form I (corresponding to the rimonabant compound disclosed in U.S. Pat. No. 5,624,941) and Form II. The two forms are disclosed as having very similar physico-chemical characteristics.

U.S. Pat. No. 5,624,941 discloses obtaining Form I by crystallizing rimonabant free base from di-isopropyl ether or methylcyclohexane (see Examples 195 and 211, respectively, thereof). Seltzman et al. (J. of Labelled Compd Radiopharm, vol. 45, (2002), p 59-70) discloses obtaining Form I by crystallizing rimonabant from diethyl ether/hexane. Dutta et al. (Med. Chem. Res., Vol. 5, (1995), p 54) discloses obtaining Form I by crystallizing rimonabant from diethyl ether/petroleum ether. The XRPD spectrum of Form I is shown in FIG. 1.

In addition, U.S. Pat. No. 5,624,941 describes obtaining a crystalline rimonabant ethanol solvate by crystallizing rimonabant from ethanol (see Example 212 thereof).

Examples 1, 3, 5 and 6 of WO 03/040105 (US 2005/0043356, EP 1446384) describe the preparation of Form II.

It would be beneficial to find other solid state forms of rimonabant, particularly solid state forms with improved tabletting properties.

### SUMMARY OF THE INVENTION

A first aspect of the present invention relates to an amorphous rimonabant free base in particulate form. The amorphous rimonabant typically contains less than 5% of any crystalline form. The particulates generally have an average particle size of less than 100 microns. The amorphous rimonabant can be formed into a pharmaceutical composition along with at least one pharmaceutically acceptable excipient.

Another aspect of the invention relates to a rimonabant substance including at least one crystalline rimonabant selected from Form III rimonabant (hydrate), Form IV rimonabant (methanol solvate), Form V rimonabant (hydrate), and Form VI rimonabant (propanol solvate). The substance may contain several forms or a single type of crystal, e.g. only Form III rimonabant, such that no other crystalline form of rimonabant is detected by XRPD. The rimonabant substance can be formulated into a pharmaceutical composition along with at least one pharmaceutically acceptable excipient.

Another aspect of the invention relates to a process for making an amorphous rimonabant, the process including desolvating a particulate rimonabant hydrate, solvate, or both, to form the amorphous rimonabant in particulate form. The desolvation step may include heating said hydrate or solvate to a temperature at least 60° C., preferably under diminished pressure. The hydrate or solvate is preferably a rimonabant ethanol solvate.

Another aspect of the invention relates to a process for making crystalline rimonabant Form III, the process including crystallizing rimonabant base from a solvent containing a mixture of water with an water-miscible organic solvent, e.g. methanol or acetone.

Another aspect of the invention relates to a process for making crystalline rimonabant Form III, the process including stirring a suspension of rimonabant hydrochloride in a solvent containing water.

### BRIEF DESCRIPTION OF DRAWINGS

FIG. 1 is an XRPD pattern of rimonabant Form I;

FIG. 2 is an XRPD pattern of amorphous rimonabant base;

FIG. 3 is an XRPD pattern of rimonabant ethanol solvate;

FIG. 4 is an XRPD pattern of rimonabant hydrate Form III;

FIG. 5 is an XRPD pattern of rimonabant methanol solvate Form IV in an admixture with rimonabant hydrate Form III;

FIG. 6 is an XRPD pattern of rimonabant hydrate Form V; and

FIG. 7 is an XRPD pattern of rimonabant propanol solvate Form VI.

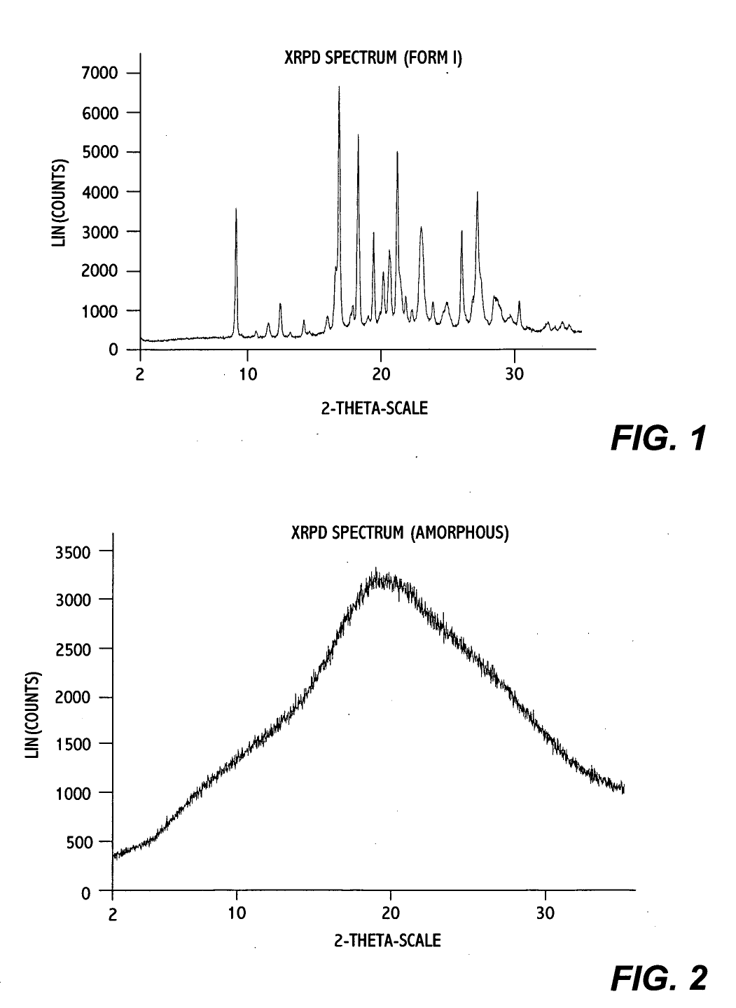

### DETAILED DESCRIPTION OF THE INVENTION

The term “rimonabant” refers to rimonabant free base, hydrates thereof and solvates thereof, but excludes rimonabant salt compounds unless otherwise indicated. The term “rimonabant substance” refers to a substance that contains at least, although not necessarily exclusively, a rimonabant as previously-defined. Thus, the term “rimonabant substance” encompasses rimonabant as a per se compound in purified or crude form as well as mixtures containing rimonabant and other compounds. The terms “particulated” and “powderized” refer to a free flowing product having a plurality of mutually distinguishable solid particles of a defined shape. The term “around” with reference to XRPD peak angles refers to plus or minus 0.2 degrees.

The present invention relates to the discovery of solid state forms of rimonabant. These solid state forms include an amorphous rimonabant base, two rimonabant hydrates (i.e., Forms III and V), and two rimonabant solvates (i.e., Forms IV and VI). These solid state forms are useful in making pharmaceutical compositions and/or as intermediates for purifying or otherwise forming a rimonabant or salt thereof.

The amorphous form of rimonabant base of the present invention is characterized in that no distinctive peaks are found on a XRPD (x-ray power diffraction) pattern thereof. Generally, less than 5% of the crystalline Form I rimonabant having characteristic XRPD peaks at angles of around 9.22°, 11.68°, 12.42°, 16.56°, 16.95°, 18.42°, 19.51°, 21.35°, 23.03°, 26.22° based on a 2-theta scale is present and more typically less than 5% of any crystalline rimonabant is present. An example of the XRPD pattern of an amorphous rimonabant is shown in FIG. 2.

In a substantial repetition of the Example 1 of EP 0658546, it was discovered that by evaporating the solution of rimonabant in a toluene/ethyl acetate mixture, a solid product may be obtained which is, according to the XRPD, an amorphous rimonabant. The resulting amorphous rimonabant is obtainable only as a foam or a glassy solid and not as a particulated or powderized solid. Accordingly, this foam/glassy solid amorphous rimonabant is not useful in a pharmaceutical product, e.g. in making tablets, and the free flowing powderized product would be more beneficial.

Quite surprisingly, a solid state amorphous rimonabant may be isolated in free flowing particulated (powderized) state by a solid/solid transformation of a solid state hydrate and/or solvate of rimonabant, such as the hydrates/solvates of the present invention and other hydrates/solvates (e.g., rimonabant ethanol solvate). This transformation does not require any solvent (i.e., it is not solvent-mediated) and it is generally accomplished by heating/drying of a powderized hydrate and/or the solvate of rimonabant base.

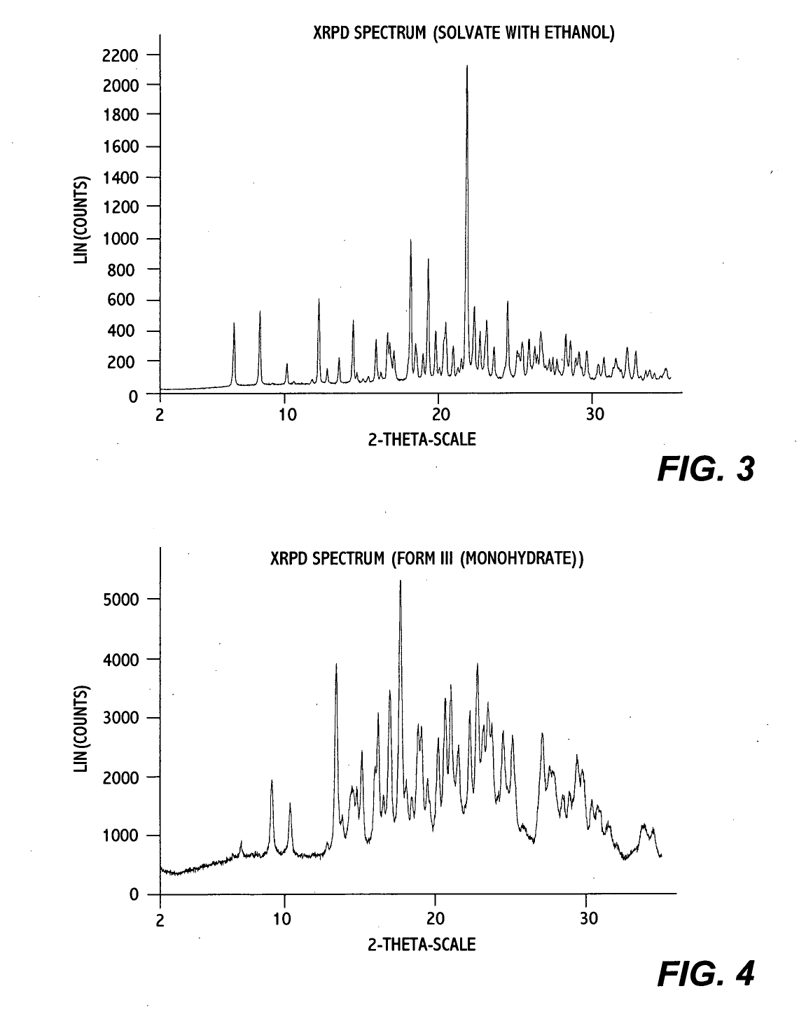

A known rimonabant solvate form, a rimonabant ethanol solvate, is disclosed in U.S. Pat. No. 5,624,941 (Example 212). According to U.S. Pat. No. 5,624,941, this ethanol solvate may be made by crystallizing rimonabant from ethanol. An example of the XRPD spectrum of the rimonabant ethanol solvate is shown in FIG. 3. It has now been discovered that the same rimonabant ethanol solvate may be obtained by a novel solvent mediated transformation, such as by suspending and stirring the anhydrous rimonabant base in a liquid comprising ethanol. For example, the liquid may be an ethanol/water mixture, e.g. having a 3:1 (V/V) ratio. Also the crystallization of rimonabant hydrochloride from an ethanol/water mixture may provide for the known ethanolate of rimonabant base, which is another process not disclosed in the prior art and indicates the instability of rimonabant hydrochloride towards solvents comprising ethanol. As described more fully below, this ethanol solvate can be used to form amorphous rimonabant.

The hydrates and solvates of the present invention (i.e., Forms III-VI), alone or in combinations thereof, are also useful for making particulate amorphous rimonabant base. Furthermore, any of these solvates and/or hydrates may be used as a pharmaceutical agent themselves or as intermediates in making rimonabant or a salt thereof such as in a purification step.

In detail, rimonabant base may be isolated in a new solid state crystalline hydrate form, designated as the Form III. Form III is characterized by the XRPD spectrum having characteristic peaks at angles of around 9.29°, 10.49°, 13.52°, 15.25°, 17.78° using a 2-theta scale. A substantially pure Form III exhibits an x-ray powder diffractogram as shown in FIG. 4. The analysis revealed that this product is a hydrate, particularly a monohydrate. The hydrated Form III is stable during storage at room temperature.

The Form III of rimonabant is obtainable by various processes. For example, the Form III may be formed in a first process by crystallizing rimonabant base from mixtures of water with an water-miscible organic solvent. The water-miscible organic solvent may be, e.g., methanol or acetone. The water of such a mixture may be present in an amount up to 80% of the total amount of the mixture. The crystallization may be spontaneous, i.e. the rimonabant base is dissolved in the solvent mixture at an enhanced temperature and the product (Form III) precipitates under cooling. Alternatively, the crystallization may be induced, i.e. the rimonabant base is dissolved in methanol and water is added as an antisolvent, whereby the product (Form III) precipitates.

Quite surprisingly, crystallizing rimonabant salt such as hydrochloride from methanol/water mixtures can also result in Form III rimonabant. In this regard, the rimonabant salt can precipitate as the base, either by addition of a neutralizing base or spontaneously, and/or can precipitate as the salt and convert to the base by a solvent mediated transformation. Accordingly, stirring a suspension of rimonabant hydrochloride in a solvent comprising water (e.g. water/methanol=30:1 V/V) at ambient temperature provides for solvent-mediated polymorphic transformation yielding the Form III of rimonabant base.

The above indicates that rimonabant hydrochloride is not stable towards solvents comprising water. A crystallization from such a solvent or stirring in such a solvent is therefore useful for making crystalline rimonabant base, particularly the hydrated Form III.

In general, only the hydrated Form III rimonabant is obtainable by a process employing water as the solvent. The other known solvent-free forms I and II are made by a process employing solely an organic solvent. The Form III is thus an advantageous form from technological and ecological reasons. The hydrated form III has the same solubility in water as the known Form I and is equally useful in pharmaceutical applications.

Rimonabant base may also be isolated in a crystalline methanol solvate form, designated as the Form IV. This methanol solvate Form IV is characterized by a XRPD spectrum having characteristic peaks at angles of around 7.61°, 9.82°, 13.38°, 15.85°, 16.26°, 18.48°, 20.00°, 23.98°, 24.87° and 32.55° using a 2-theta scale. The previously unknown crystalline methanol solvate of rimonabant (Form IV) may be obtained by crystallizing rimonabant base from a water-free methanol solvent. As methanol generally contains traces of water and/or may absorb water from the environment, the rimonabant methanolate solvate is difficult to obtain in a polymorphically pure state.

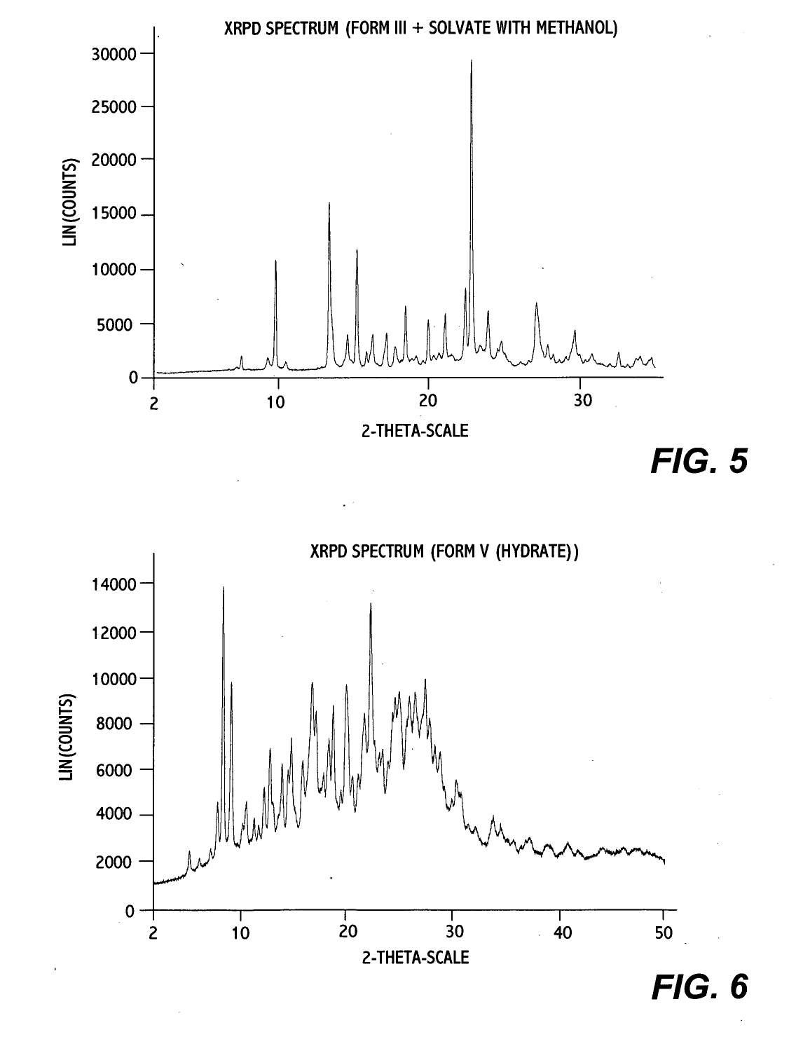

In general, a mixture of the hydrated Form III and the methanol solvate Form IV may be obtained after a crystallization of rimonabant base from a methanol solvent containing water. Mixtures of the hydrated Form III and the methanol solvate Form IV may also be obtained by crystallizing rimonabant base and rimonabant hydrochloride from methanol/water mixtures. A longer crystallization time, a slower cooling, and a lower solute concentration each generally promote the crystallization of Form III over Form IV. Furthermore, the rimonabant methanol solvate Form IV may be converted into Form III during storage at room temperature in the presence of air humidity. The methanolate Form IV is thus a thermodynamically unstable form. A mixture of Forms III and Form IV is also useful for making particulated amorphous rimonabant base. An example of a XRPD spectrum of the Form III/Form IV mixture is shown in FIG. 5.

Rimonabant base may also be isolated in a crystalline hydrate form, designated as the Form V. Form V is characterized by the following XRPD having characteristic peaks at angles of around 5.33°, 6.29°, 8.00°, 10.35°, 10.68°, 11.41°, 12.90°, 13.99°, 15.94°, 16.85°, 17.20°, 18.82° and 26.58° using a 2-theta scale. Form V may be a monohydrate having a different crystalline lattice from Form III. An example of the XRPD spectrum of the Form V is shown in FIG. 6. Form V may be obtained by solidifying/freezing a solution of rimonabant base in acetonitrile, followed by melting the solidified/frozen mixture at ambient temperature in air.

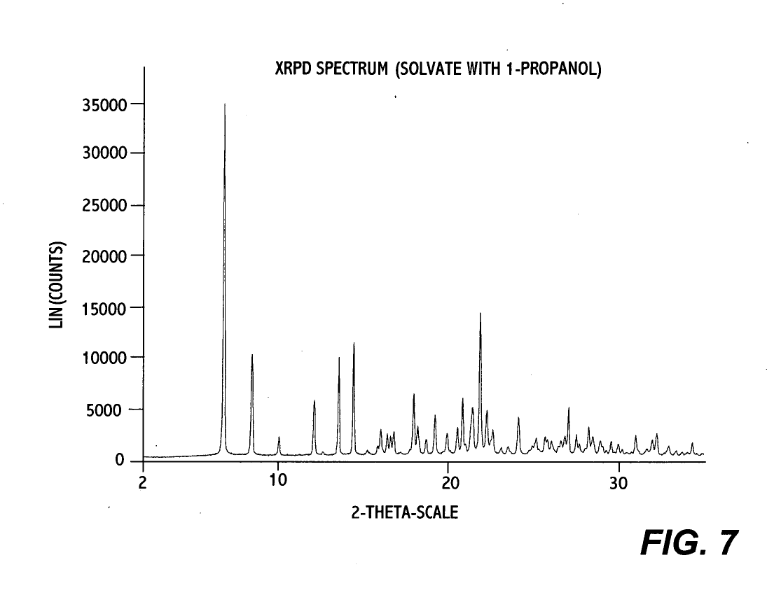

Rimonabant base may also be isolated in a propanol solvate form, designated as the Form VI. Form VI is characterized by the following XRPD having characteristic peaks at angles of around 6.73°, 8.36°, 9.92°, 11.98°, 13.45°, 14.34°, 18.14°, 21.43°, 21.88°, 24.13°, 31.03° and 32.28° using a 2-theta scale. An example of the XRPD spectrum is shown in FIG. 7. Form VI is sufficiently stable during storage at room temperature. It should be noted that 2-propanol does not provide for a crystalline solvate of rimonabant. Instead, the known Form I of the crystalline anhydrous rimonabant is provided after a crystallization from 2-propanol. Form VI may be obtained by crystallizing rimonabant base from a 1-propanol solvent. For example, the crystallization may be induced by a slow evaporation of the 1-propanol solvent.

The conversion of any of the above hydrates and/or solvates, as well as mixtures thereof, to the particulated amorphous rimonabant base may be performed by a heating (drying) thereof at an enhanced temperature (e.g., higher than room temperature), preferably at diminished pressure (e.g., lower than atmospheric pressure). The transformation into the particulated amorphous rimonabant base may be accomplished by the fact that the crystalline lattice of any of the solvates and/or hydrates is thermally unstable and may collapse at an enhanced temperature without any further recrystallization into the most thermodynamically stable form of rimonabant, the Form I. Accordingly the Form I cannot apparently be converted into the amorphous form by such a simple transformation.

The fastest loss of the bound solvent in combination with amorphization occurs for the ethanol solvate, where the heating/drying of the product takes place at 60° C. or more, generally 60-80° C. Accordingly, the ethanol solvate can be used to obtain powderized amorphous rimonabant base. For other hydrates/solvates, e.g., for the methanolate Form IV, propanolate Form VI, hydrate Form III or hydrate Form V, a temperature higher than 80° C. is preferable to facilitate the conversion into the particulate amorphous rimonabant base. The Form VI with the 1-propanol solvent appears to be the most stable of these other hydrates and solvates. Dehydration of Form III might also give some Form I in the amorphous rimonabant base product, but generally less than 5% of Form I is formed (i.e., 0% to less than 5%). Regardless, produced particulated amorphous rimonabant base according to the present invention is still a free flowing powder, not a glass or foam, which is convenient for further use, e.g., for making pharmaceutical compositions. Conveniently, the average particle size of the particulated amorphous rimonabant base may be less than about 100 microns (such as less than about 50 microns or less than about 20 microns), which may be achieved by a selection of the particle size of the starting material, as the desolvation is not accomplished by any particular association of the original solid crystals.

Amorphous rimonabant base or one or more hydrates/solvates of rimonabant can be formulated into various pharmaceutical compositions with one or more pharmaceutically acceptable excipients. The pharmaceutical composition can be a unit dosage form, such as a solid oral dosage form (i.e. tablet or capsule), or bulk precursor thereof, such as a pre-blended mixture ready for further blending/addition of ingredients or a blend ready for tabletting or filling into capsules.

The excipient may be a pharmaceutically acceptable carrier or diluent, such as one or more of calcium phosphates, microcrystalline cellulose, hydroxypropyl methylcellulose, lactose, and starches, but is not limited thereto. A polymer that is able to form a molecular dispersion with rimonabant can also be used as an excipient. An example of such a polymer is polyvinylpyrrolidone or hydroxypropylmethylcellulose phthalate. Such a dispersion can be formed by methods well known in the art, for example, dissolving the amorphous rimonabant and the polymer in a suitable solvent and evaporating the solvent. Other excipients include fillers, binders, lubricants, disintegrants, preservatives, pH-adjustors, colorants, etc.

The pharmaceutical compositions are preferably formulated into tablets. The tablets may be monolithic tablets, i.e. tablets that upon ingestion do not disintegrate into a plurality of smaller units from which the active ingredient is finally released, or may be disintegrable tablets. The tablets may be produced by any standard tabletting technique, e.g. by wet granulation, dry granulation, or direct compression. The tabletting methods that do not employ a solvent (“dry processes”) are preferable. The tablet compositions may be further coated by a film coat. The film coat may protect the tablet against the environment (light, air, moisture) during storage and handling. Any conventional film coat may be used.

Alternatively, rimonabant pharmaceutical compositions can be filled into capsules. Generally, the process comprises blending the active substance and excipients in one or more mixing or blending steps and then filling the blend into capsules.

The pharmaceutical compositions of the present invention typically contain particulated amorphous rimonabant base or Form III rimonabant, which are preferably free from other solid-state forms of rimonabant, although other forms can be used. Thus, the pharmaceutical composition may be substantially free of the rimonabant Form I, e.g., containing less than 2%, more preferably less than 1%, relative to the sum of all forms.

The pharmaceutical compositions of the present invention are normally formulated into a unit dosage forms, such as the above-described tablets or capsules. In a unit dosage form, the total amount of rimonabant present, regardless of form, is effective for providing the desired therapeutic effect, such as an anti-obesity effect, to a mammal. The unit dose may be a single tablet, one half of a tablet, or two or more tablets taken at essentially the same time or in the same administration. Unit dose in capsule form may comprise one or more capsules. The particulated amorphous rimonabant base can be used to treat a mammal in need thereof (e.g., obese mammals) by administering an effective amount of the rimonabant.

The above disclosed novel hydrates and/or solvates of rimonabant may be also formulated into the pharmaceutical formulations and used at the similar conditions as disclosed above for the particulated amorphous rimonabant base.

The invention is further illustrated by the following examples.

### Preparation 1

### Amorphous Rimonabant (Similar to Example 1 of EP 0658546)

2.0 g of anhydrous rimonabant base was dissolved in 200 ml of toluene/ethyl acetate (9:1 V/V) at R.T. and by means of stirring. The solution was filtered over a P3-glass filter (reduced pressure). The filtrate was evaporated under vacuum to dryness, yielding an off-white foam. The yield was not determined.

DSC: only weak effects, no melting.

TGA: about 7.5 m % mass loss below 260° C.

XRPD: no diffraction peaks, only “amorphous hump”.

### Preparation 2

### Rimonabant Hydrate Form III by Recrystallization of Rimonabant Hydrochloride (Corresponding to a Modification of_Example 2 of WO2006/087732)

5.0 g of rimonabant hydrochloride was dissolved in 30 ml of methanol around 45° C. while stirring. To the warm solution, 60 g of crushed ice was added. Rapid precipitation took place, while the temperature of the suspension dropped to about −14° C. The suspension was stirred for about 40 minutes during which the temperature slowly rose (not measured). In addition, the suspension became very thick. The solid was isolated by filtration over a P3-glass filter (reduced pressure), taking about 1 hour. The solid was air-dried overnight under ambient conditions. An off-white, crystalline powder with lumps was obtained. The yield was 4.30 g.

TGA: About 3.89 m % below 105° C. (about 1.04 eq. of water).

XRPD: rimonabant base, form III.

### Preparation 3

### Rimonabant Ethanol Solvate

3.5 g of rimonabant base, vacuum dried at 80° C., was dissolved in 20 ml of ethanol at reflux and by means of stirring. Reflux was maintained for about 10 minutes. Then, the solution was slowly cooled to R.T., during which crystallization occurred, and left at R.T. for about 2.5 hours. The solid was isolated by filtration over a P3-glass filter (reduced pressure) and air dried overnight under ambient conditions. An off white, crystalline powder with lumps was obtained. The yield was 3.28 g.

TGA: about 8.1 m % of mass loss below 210° C., corresponding to 0.89 eq. of ethanol.

XRPD: See FIG. 3

NMR: About 0.8 equivalent of ethanol detected.

### EXAMPLE 1

### Rimonabant Hydrate Form III from Methanol/Water

0.5 g of rimonabant base was dissolved in 10 ml of methanol around 50° C. and by means of stirring. The solution was filtered over a P3-glass filter (reduced pressure) and heated up to 50° C. again. To the warm solution, removed from the oil bath.

35 g of crushed ice was added, while being stirred. Rapid precipitation took place, while the temperature of the suspension dropped to about −11° C. The solid was immediately isolated by filtration over a P3-glass filter (reduced pressure), washed with some water and air dried under ambient conditions for about 3 days. An off-white, crystalline powder with lumps was obtained. The yield was 330 mg.

TGA: about 3.26 m % below 105° C. (about 0.87 eq. of water).

XRPD: See FIG. 4

### EXAMPLE 2

### Rimonabant Hydrate Form III from Acetone/Water

0.5 g of rimonabant base was dissolved in 25 ml of acetone at R.T. and by means of stirring. To the solution, stirred at R.T.,

100 ml of demi-water was added. As a result, crystallization took place. The suspension was stirred at R.T. for an additional 1.5-2 hours, during which small particles coagulated into larger particles. The solid was isolated by filtration over a P3-glass filter (reduced pressure) and air dried under ambient conditions for 3 days. A white, crystalline powder with a yield of 430 mg was obtained.

TGA: about 3.8 m % of mass loss below 125° C., corresponding to 1.0 eq. of water.

XRPD: form III.

### EXAMPLE 3

### Rimonabant Hydrate Form III from Methanol

0.5 g of rimonabant base was dissolved in 5 ml of methanol at reflux and by means of stirring. Reflux was maintained for 15-20 minutes. Then, the hot solution was slowly cooled to R.T. by stepwise lowering the oil bath temperature (stirring continued). The solution was stirred overnight at R.T., during which crystallization occurred. The solid was isolated by filtration over a P3-glass filter (reduced pressure) and air dried under ambient conditions for 3 days. A white, fine powder with lumps was obtained. The yield was 270 mg.

TGA: about 3.8 m % of mass loss below 120° C., corresponding to 1.0 eq. of water.

XRPD: form III. No peaks of the methanolate present.

### EXAMPLE 4

### Mixture of Forms III and IV from Methanol

0.5 g of rimonabant base was dissolved in 5 ml of methanol at reflux. The hot solution was allowed to cool to R.T. and left overnight at R.T. in a closed flask. No crystallization occurred. Then, the flask was scratched with a spatula, inducing sudden crystallization. After an additional 4 hours at R.T., the solid was isolated by filtration over a P3-glass filter (reduced pressure) and air dried under ambient conditions for about 1 day. Bunches/aggregates of fine needles or fibres, like cotton wool, were present. The yield was 360 mg.

DSC: (melting) endotherm around 94-102° C. with a broad shoulder/drift above 70° C.

TGA: about 5.8 m % of mass loss below 220° C.

XRPD: Peaks for Form III+extra peaks attributed to Form IV

NMR: About 0.25 eq. of methanol were detected.

### EXAMPLE 5

### Rimonabant Hydrate Form V

0.5 g of rimonabant base, form I was dissolved in 5 ml of acetonitrile at reflux. The hot solution was filtered over a hot P3-glass filter (reduced pressure) into a cold round bottomed flask of 100 ml, which was kept at −78° C. The solution immediately solidified as acetonitrile has a melting point of −44° C. The solidified solution was left just above the CO₂-ice/acetone bath to allow slow warming up. During melting of the solvent, crystallization of the drug substance occurred. After about 1.5-2 hours, the solid was isolated by filtration over a P3-glass filter (reduced pressure) and air dried under ambient conditions for 4 days. An off white, crystalline powder with a good flowability was obtained. The yield was 310 mg.

TGA: about 3.3 m % of mass loss below 120° C., corresponding to 0.87 eq. of water.

XRPD: See FIG. 6

### EXAMPLE 6

### Rimonabant Propanol Solvate Form VI

0.5 g of rimonabant base was dissolved in 5 ml of 1-propanol at reflux. The hot solution was allowed to cool to R.T. and left overnight at R.T. in a closed flask. No crystallization occurred. Then, the solution was kept at 4° C. for an additional 1 day, but again no crystals were formed. Subsequently, the solution was left at R.T. in an open flask with cotton wool in the neck to allow very slow evaporation of solvent. After 5 days, still no crystals present. Finally, the cotton wool was removed and the solution was left at air for an additional 2 days, during which crystallization occurred. White, fluffy aggregates of thin needles/fibres were present. The yield was 540 mg, which is more than 100%, likely due to the presence of (bound) solvent.

TGA: two large steps below 220° C. (3.86 m % and 7.31 m %). Both steps together correspond to 1 equivalent of 1-propanol.

XRPD: Form VI.

NMR: 1 equivalent of 1-propanol found.

### EXAMPLE 7

### Rimonabant Form III and Form IV from Rimonabant Hydrochloride

0.51 g of rimonabant hydrochloride was dissolved in 10 ml of methanol at R.T. The solution was filtered over a P3 glass filter (reduced pressure) as some “haze” was visible. To the solution, stirred at R.T.,

50 ml of demi-water was added dropwise. As a result, immediate precipitation took place.

The solid was isolated by filtration over a P3-glass filter (reduced pressure) and air-dried overnight under ambient conditions. A white, crystalline powder was obtained. The yield was 400 mg.

Analysis: IR and NMR pointed to the free base of rimonabant.

IR and XRPD indicated the mixture of forms III and IV.

### EXAMPLE 8

### Rimonabant Methanolate (Form IV)/Hydrate (Form III) Mixture from Rimonabant Hydrochloride

0.5 g of rimonabant hydrochloride was dissolved in 60 ml of methanol/water (2:1 V/V) at reflux. The hot and clear solution was allowed to cool to R.T. and left at R.T. for about 3 days, during which crystallization occurred. The crystals were isolated by filtration over a P3-glass filter (reduced pressure) and air-dried under ambient conditions for about 3 days. White to colourless needles/plates, often in bunches were obtained. The yield was not determined.

TGA: two clear steps below 175° C. (4.71 m % and 1.02 m % respectively, max. 0.9 eq. of methanol or 1.5 eq. of water).

XRPD: The mixture of Forms III and IV

NMR: the free base has been formed, about 0.667 eq. methanol present.

### EXAMPLE 9

### Amorphous Rimonabant Base, Particulated

About 100 mg of rimonabant hydrate Form III was vacuum dried at 60° C. for 16 hours and further dried at 80° C. and under vacuum for 16 hours. A powderized material was obtained.

XRPD revealed mainly particulated amorphous rimonabant base with a small amount of rimonabant hydrate form III.

### EXAMPLE 10

### Amorphous Rimonabant Base, Particulated

3.3 g of the rimonabant ethanol solvate obtained from preparation 3 was vacuum dried at 60° C. for about 1 day and subsequently vacuum dried at 80° C. for an additional day, yielding amorphous rimonabant base in a powderized state.

DSC: only weak effects, no melting.

TGA: about 0.3 m % mass loss below 60° C.

XRPD: no diffraction peaks

Each of the patents, patent applications, and journal articles mentioned above are incorporated herein by reference. The invention having been described it will be obvious that the same may be varied in many ways and all such modifications are contemplated as being within the scope of the invention as defined by the following claims.

We claim:

1. Amorphous rimonabant free base in particulate form.

2. The amorphous rimonabant of claim 1 comprising less than 5% of any crystalline form.

3. The amorphous rimonabant of claim 1 or 2 wherein said particulates have an average particle size of less than 100 microns.

4. A pharmaceutical composition comprising said amorphous rimonabant according to claim 1 and at least one pharmaceutically acceptable excipient.

5. A rimonabant substance, comprising at least one crystalline rimonabant selected from the group consisting of Form III rimonabant (hydrate), Form IV rimonabant (methanol solvate), Form V rimonabant (hydrate), and Form VI rimonabant (propanol solvate).

6. The rimonabant according to claim 5, which comprises at least crystalline rimonabant Form III.

7. The rimonabant according to claim 6, wherein said substance exhibits an XRPD pattern that includes the following peaks at 2θ: 9.29°, 10.49°, 13.52°, 15.25°, and 17.78° each +/−0.2 degrees.

8. The rimonabant according to claim 7, wherein no other crystalline form of rimonabant is detected by XRPD.

9. The rimonabant according to claim 6, which further comprises crystalline rimonabant Form IV.

10. The rimonabant according to claim 5, which comprises crystalline rimonabant Form IV (methanol solvate) and wherein said substance exhibits an XRPD pattern that includes the following peaks at 2θ: 7.61°, 9.82°, 13.38°, 15.85°, 16.26°, 18.48°, 20.00°, 23.98°, 24.87° and 32.55°, each +/−0.2 degrees.

11. The rimonabant according to claim 5, which comprises crystalline rimonabant Form VI (propanol solvate) and wherein said substance exhibits an XRPD pattern that includes the following peaks at 2θ: 6.73°, 8.36°, 9.92°, 11.98°, 13.45°, 14.34°, 18.14°, 21.43°, 21.88°, 24.13°, 31.03° and 32.28°, each +/−0.2 degrees.

12. The rimonabant according to claim 5, which comprises crystalline rimonabant Form V (hydrate) and wherein said substance exhibits an XRPD pattern that includes the following peaks at 2θ: 5.33°, 6.29°, 8.00°, 10.35°, 10.68°, 11.41°, 12.90, 13.99°, 15.94°, 16.85°, 17.20°, 18.82° and 26.58, each +/−0.2 degrees.

13. A pharmaceutical composition comprising the rimonabant substance according to claim 5 and at least one pharmaceutically acceptable excipient.

14. A process for making the amorphous rimonabant, which comprises desolvating a particulate rimonabant hydrate, solvate, or both, to form amorphous rimonabant in particulate form.

15. The process according to claim 14, wherein said desolvation step comprises heating said hydrate or solvate to a temperature at least 60° C., optionally under diminished pressure.

16. The process according to claim 14, wherein the hydrate or solvate is a rimonabant ethanol solvate.

17. A process for making crystalline rimonabant Form III, which comprises crystallizing rimonabant base from a solution containing rimonabant or its salt dissolved in a solvent comprising a mixture of water and a water-miscible organic solvent.

18. The process according to claim 17, wherein said solvent comprises methanol or acetone.

19. A process for making crystalline rimonabant Form III, which comprises stirring a suspension of rimonabant hydrochloride in a solvent comprising water.

20. The process according to claim 19, wherein said solvent comprises water and methanol.
